(19) United States
(12) Patent Application Publication
(10) Pub. No.: US 2018/0280574 A1
(43) Pub. Date: Oct. 4, 2018

(54) Extracellular matrix compositions with bactericidal or bacteriostatic characteristics useful for protecting and treating patients with bacterial infections

(71) Applicants: ACell, Inc., Columbia, MD (US); University of Pittsburgh, Pittsburgh, PA (US)

(72) Inventors: Thomas Wayne Gilbert, Ellicot City, MD (US); Yuanpu Di, Wexford, PA (US)

(73) Assignees: ACell, Inc., Columbia, MD (US); University of Pittsburgh, Pittsburgh, PA (US)

(21) Appl. No.: 15/935,707

(22) Filed: Mar. 26, 2018

Related U.S. Application Data
(60) Provisional application No. 62/479,888, filed on Mar. 31, 2017.

(51) Int. Cl. A61L 27/36; A61P 11/04; A61P 11/06; A61P 31/04; A61K 9/00; A61K 35/12; A61L 27/38

(52) U.S. Cl. CPC A61L 27/3633; A61P 11/04; A61P 11/06; A61P 31/04; A61L 2430/40; A61K 35/12; A61L 27/3691; A61L 27/3804; A61K 9/0075

(57) ABSTRACT

Described is a formulation and method for reducing and treating bacterial infections in humans and animals with digested or non-digested extracellular matrix materials derived from non-epithelial and epithelial tissues.

### TECHNICAL FIELD

The invention described herein is directed to compositions, methods of making and methods of use for treating bacterial infections in humans and animals.

### RELATED APPLICATIONS

This application claims priority to and benefit of U.S. provisional application No. 62/479,888, filed Mar. 31, 2017, incorporated by reference herein in its entirety for all intents and purposes.

### BACKGROUND

Bacterial infection frequently compromises the healing process of patients' burns, chronic wounds, and other bacterial infections of tissues and organs, pneumonia, for example. Yet, commonly used prophylactic antibiotics such as topical silver sulfadiazine, are associated with an increase in the rates of burn wound infection, failed therapy, and an increased length of hospital stay. Ideally, it would be advantageous to treat burn wounds with local and systemic bacterial infections with a composition in vivo that possesses bacterial growth inhibitory activity. In this instance, treatment with this composition preferably would allow for reduction or elimination of the need for additional antibiotic application. The compositions and methods for achieving the above advantages are described below.

Staphylococcus aureus is a gram-positive coccal bacterium that is frequently found in the nose, respiratory tract, and on the skin of humans and is one of the common causes of infections after injury or surgery. Due to wide spread use of currently available antibiotics and bacterial evolution, antibiotic resistant gram-positive Staphylococcus aureus, gram-negative Pseudomonas aeruginosa and Klebsiella pneumoniae strains have emerged in recent years.

Methicillin-resistant Staphylococcus aureus (MRSA) is any strain of Staphylococcus aureus that has developed resistance to beta-lactam antibiotics, which include the penicillins (methicillin, dicloxacillin, oxacillin, etc.) and the cephalosporins. Strains unable to resist these antibiotics are classified as methicillin-susceptible Staphylococcus aureus, or MSSA. The most significant development regarding MRSA's overall impact on human health has been the increasing threat it poses as a community-acquired infection. Over the past two decades, MRSA has gone from being a nosocomial infection, with 65% of MRSA cases arising in a hospital setting and affecting ailing patients, to a predominantly community-acquired illness infecting otherwise healthy individuals with frequently fatal outcomes. An improved method for preventing and treating such infections in humans and animals is needed.

Pseudomonas aeruginosa (PA) is a type of gram-negative rod-shaped bacteria that causes a variety of infectious diseases in animals and humans. It is increasingly recognized as an emerging opportunistic pathogen of clinical significance, often causing nosocomial infections. P. aeruginosa infection is a life-threatening disease in immune-comprised individuals, and its colonization has been an enormous problem in cystic fibrosis patients. Several epidemiological studies indicate that antibiotic resistance is increasing in clinical isolations of P. aeruginosa because it can develop new resistance after exposure to antimicrobial agents.

Klebsiella (KP) is also a common Gram-negative pathogen causing community-acquired bacterial pneumonia and 8% of all hospital-acquired infections. Lung infections with Klebsiella pneumoniae are often necrotic. The observed mortality rates of community-acquired Klebsiella pneumoniae range from 50% to nearly 100% in alcoholic patients. Carbapenem-resistant enterobacteriaceae (CRE) including Klebsiella species are among the bacteria of urgent threats based on a CDC report, while MRSA and PA are both categorized as serious threats.

The inventions described herein include compositions and methods that address these problems and are applicable where bacterial contamination or infection warrants alternative treatments.

Scaffold materials, especially those derived from naturally occurring extracellular matrix of epithelial tissues elicit an integration response when applied in a patient. The extracellular matrix (ECM) consists of a complex mixture of structural and functional macromolecules that is important during growth, development, and wound repair. Scaffold materials derived from ECMs include but are not limited to non-epithelial derived ECMs, small intestinal submucosa (SIS), urinary bladder submucosa (UBS), liver (L-ECM) and urinary bladder matrix (UBM).

Urinary bladder matrix is a biologically-derived scaffold extracellular matrix material described in U.S. Pat. No. 6,576,265, incorporated by reference herein in its entirety for all purposes, which consists of a complex mixture of native molecules that provide both structural and biological characteristics found in the epithelial basement membrane and other layers of epithelial tissues, such as, but not limited to the urinary bladder. UBM has been used as an effective scaffold to promote site-appropriate tissue formation, referred to as constructive remodeling, in a variety of body systems. UBM scaffolds provide a scaffold for tissue as it is completely resorbed by the body. Due to the composition of the scaffold and degradation kinetics, the host response to UBM has been characterized by an adaptive immune response, with a prevalence of T helper cells and M2 macrophages at the site of remodeling. The degradation of UBM has been shown to result in the released peptide fragments that are capable of facilitating constructive remodeling.

### SUMMARY OF THE INVENTION

Surprisingly, in the studies described herein, an exemplary ECM derived from the porcine urinary bladder, specifically urinary bladder matrix (UBM) was identified as exhibiting bacterial activity in vitro and in vivo toward a lab strain of MSSA and appreciable anti-biofilm activity against multiple clinical MRSA, PA and KP isolates. A mouse model was used to study the potential usefulness of ECMs such as UBM in preventing, lessening, and/or eliminating bacterial infection in humans and animals. Both gram positive bacteria (GPB) MSSA- and MRSA- and gram negative bacteria (PA)-induced respiratory infection in mice result in significantly increased lung bacterial burden that is accompanied by increased recruitment of neutrophils and elevated pro-inflammatory cytokines and chemokines. However, exogenous administration of UBM digest through intra-tracheal instillation protected the inoculated mice from severe lung infection by significantly decreasing the bacterial burden and by attenuation of the bacterial cytokine/chemokine secretion. Furthermore, water reconstituted pre-formulated digested UBM that was kept at room temperature for prolonged periods of time, as well as an undigested particulate form of UBM, can similarly achieve the protected function of UBM against GPB- and GNB-induced infection to provide an off-the-shelf and easily accessible resource to minimize and treat bacterial infection.

Taken together, the results of the studies described below support the use of UBM as an alternative or an adjunct to known therapies for the attenuation if not elimination of GPB- and GNB-induced infection in mammals including but not limited to pneumonia, wounds, burns, persistent infections of the skin, comminuted bone fractures, cystitis, cellulitis, local and systemic bacterial infections, and nosocomial infections in humans and animals.

In one aspect, the inventions described herein are directed to methods for the treatment of bacterial infections such as, but not limited to, a respiratory infection in a patient, comprising, administering to the patient via a suitable route, for example, but not limited to, an airway, an effective dose of a non-cross-linked, micronized powder obtained from a devitalized native extracellular matrix material, preferably processed at room temperature. The devitalized native extracellular matrix is selected from the group consisting of non-epithelial tissue, UBM, SIS, and UBS.

In one embodiment of the invention, the micronized powder is non-enzymatically treated and may be stored at room temperature for a prolonged length of time, such as, but not limited to as long as four weeks, two months, six months, one year, two years, five years and still retains its efficacy for the treatment of animal and human bacterial infections.

The bacterial infection treated by the above micronized powder may be caused by gram positive bacteria, such as, but not limited to bacteria consisting of Staphylococcus aureus related bacteria, or gram negative bacteria, such as, but not limited to bacteria selected from the group consisting of Pseudomonas aeruginosa, and Klebsiella pneumoniae and related bacteria.

The respiratory infection may be localized in airways including the lung, and the route of administration includes routes via inhalation, via a spray or a respirator, intra-nasal instillation or by an intra-tracheal route. Alternatively, the route of administration comprises lavaging the airways of the patient with the micronized ECM particle in a buffer solution.

In another aspect, the invention is directed to a composition, comprising

a reconstituted material in a buffer solution comprising enzymatically or non-enzymatically digested, micronized powder obtained from a devitalized extracellular matrix material including epithelial basement membrane, said reconstituted material comprising one or more native components of the extracellular matrix. The buffer may be selected from any physiological buffer such as, but not limited to, buffered saline.

In another aspect, the invention is directed to methods for reducing bacterial biofilm formation in a patient infected with a bacteria by administering to the patient a micronized, devitalized extracellular matrix of an epithelial tissue comprising bactericidal activity against one or more bacteria in a therapeutically effective dose. The one or more bacteria may be selected from, but not limited to the group consisting of MSSA-, MSRA-Staphylococcus aureus, Klebsiella pneumoniae and Pseudomonas aeruginosa. The treatment may prevent, lessen or eliminate the bacterial infection.

In yet another aspect, the invention is directed to methods to protect a mammal from a bacterial-induced infection by providing a reconstituted material comprising a micronized powder in a buffer solution obtained from a devitalized extracellular matrix material of an epithelial or non-epithelial tissue, the reconstituted material comprising one or more native components of the extracellular matrix, and administering the material in a therapeutically effective dose by a route selected from but not limited to the group consisting of intra-tracheal instillation, intra-nasal inhalation, spray, transoral inhalation, topical application, lavage, and combinations thereof.

### BRIEF DESCRIPTION OF THE DRAWINGS

The drawings generally place emphasis upon illustrating the principles of the invention.

FIGS. 1A-H graphically illustrate pepsin-digested UBM increased antibacterial activity against MSSA as compared to PBS-extracted UBM supernatant.

FIG. 1A graphically illustrates inhibition of MSSA growth by PBS-extracted UBM supernatant.

FIG. 1B graphically illustrates growth of MRSA in the presence of PBS-extracted UBM supernatant.

FIG. 1C graphically illustrates growth of Pseudomonas aeruginosa (PAO1) in the presence of PBS-extracted UBM supernatant.

FIG. 1D graphically illustrates growth of Klebsiella pneumoniae in the presence of PBS-extracted UBM supernatant.

FIG. 1E graphically illustrates inhibition of MSSA growth by enzymatically digested UBM.

FIG. 1F graphically illustrates growth of MRSA in the presence of enzymatically digested UBM.

FIG. 1G graphically illustrates growth of Pseudomonas aeruginosa (PAO1) in the presence of enzymatically digested UBM.

FIG. 1H graphically illustrates growth of Klebsiella pneumoniae in the presence of enzymatically digested UBM. The measurement of optical density represents the bacterial growth in culture media. Results were obtained from three independent experiments.

FIGS. 2A-D graphically illustrate that instillation of digested UBM (10 mg/kg intra-tracheally (i.t.) into wild-type FVB/NJ mouse lung does not cause pulmonary toxicity.

FIG. 2A illustrates total inflammatory cells and differential cell counts in PBS and UBM-treated mouse lung.

FIG. 2B illustrates total protein in BAL in PBS and UBM-treated mouse lung.

FIG. 2C illustrates expression of inflammation-associated genes in PBS and UBM-treated mouse lung.

FIG. 2D illustrates expression of epithelial cell-associated genes in PBS and UBM-treated mouse lung. The results illustrated in FIGS. 2A-D suggest that UBM does not cause pulmonary toxicity. Results are mean±SEM from two independent experiments; n=5 mice for each group.

FIGS. 3A-D graphically illustrate that UBM treated mice are protected against MSSA-induced respiratory infection.

FIG. 3A graphically illustrates CFU in lung, BAL, and total lung burden (BAL plus lung homogenate) in MSSA infected PBS treated compared to UBM treated mice.

FIG. 3B graphically illustrates differential cell counts in MSSA infected PBS treated mice compared to UBM treated mice.

FIG. 3C graphically illustrates expression of inflammation-related genes in MSSA infected PBS treated mice compared to UBM treated mice.

FIG. 3D graphically illustrates the expression of epithelial cell associated genes in MSSA infected PBS treated mice compared to UBM treated mice. Results are mean±SEM from three independent experiments; n=4-6 mice for each treatment group. *p<0.05, **p<0.01 for UBM-treated to PBS-treated comparisons.

FIGS. 4A-D graphically illustrate UBM treatment protects mice from MRSA-induced respiratory infection.

FIG. 4A graphically illustrates that UBM treatment resulted in significantly decreased CFU in BAL, lung, and total lung burden (BAL plus lung homogenate) in age-matched wild-type FVB/NJ mice intranasally (i.n.) inoculated with 2×10⁶ CFU MRSA (USA300) per mouse; MRSA infected PBS treated mice compared to UBM treated mice.

FIG. 4B graphically illustrates differential cell counts in MRSA infected, PBS treated mice compared to UBM treated mice.

FIG. 4C graphically illustrates expression of inflammation-related genes in MRSA infected, PBS treated mice compared to UBM treated mice.

FIG. 4D graphically illustrates expression of epithelial cell-associated genes in MRSA infected PBS treated mice compared to UBM treated mice. Results are mean±SEM from three independent experiments; n=4-6 mice for each treatment group. *p<0.05, **p<0.01 for UBM-treated to PBS-treated comparisons.

FIGS. 5A-D graphically illustrate UBM significantly inhibits biofilm formation of GPB (MSSA and MRSA) and GNB (PA and KP) bacteria.

FIG. 5A illustrates biofilm formation of MSSA after treatment with different concentrations of UBM.

FIG. 5B illustrates biofilm formation of MRSA after treatment with different concentrations of UBM.

FIG. 5C illustrates biofilm formation of PA after treatment with different concentrations of UBM.

FIG. 5D illustrates biofilm formation of KP after treatment with different concentrations of UBM. Results are mean±SEM from three independent experiments. ***p<0.005, and ****p<0.001 for the comparison between the treatment group to the control group.

FIGS. 6A-D graphically illustrate UBM treatment protects mice from P. aeruginosa-induced respiratory infection.

FIG. 6A graphically illustrates CFU in BAL, lung, and total lung burden (BAL plus lung homogenate) at 15 h after P. aeruginosa infection in UBM vs. PBS treated mice.

FIG. 6B graphically illustrates differential cell counts at 15 h after P. aeruginosa infection in UBM treated mice vs. PBS treated mice.

FIG. 6C graphically illustrates expression of inflammation-related genes at 15 h after P. aeruginosa infection in UBM treated mice vs. PBS treated mice.

FIG. 6D graphically illustrates expression of epithelial cell-associated genes at 15 h after P. aeruginosa infection in UBM vs. PBS treated mice treated mice. The results illustrated in FIGS. 6A-D showed no statistical difference between UBM-treated and PBS-treated mice at 15 h post-infection. Results are mean±SEM from three independent experiments; n=5 mice for each treatment group. *p 21 0.005, and **p<0.01 for UBM-treated to PBS-treated comparisons.

FIGS. 7A-B graphically illustrate pre-formulated UBM (PF-UBM) shows comparable bioactivity to freshly digested UBM (FD-UBM).

FIG. 7A illustrates in vitro anti-biofilm activity of UBM against MSSA (ATCC#49775) and MRSA (USA300).

FIG. 7B illustrates in vivo antibacterial activity by bacterial CFU in mouse BAL, lung, total lung burden (BAL plus lung homogenate), and spleen at 15 h after MRSA infection. The results illustrated in FIGS. 7A-7B showed no statistical difference between pre-formulated (PF-UBM) and freshly digested (FD-UBM) UBM in their protection against MRSA infection. Both PF-UBM and FD-UBM showed significant protection against MRSA-induced bacterial infection in mice. Results are mean±SEM from three independent experiments; n=5 mice for each group. Following one-way analysis of variance (ANOVA), post hoc comparisons were made using the Dunnett's multiple comparison test when the P-value was significant (P<0.05). *p<0.05, **p<0.01, ***p<0.005, and ****p<0.001 for the comparison between groups.

FIGS. 8A-C graphically illustrate that exogenously administered pre-formulated UBM significantly attenuates inflammatory response that was induced by respiratory MRSA infection.

FIG. 8A illustrates gene expression of cytokines and chemokines in MRSA-infected mice comparing FD-UBM, PD-UBM and PBS-treated mice lungs.

FIG. 8B illustrates protein secretion of cytokines and chemokines in mice BAL in MRSA-infected mice comparing FD-UBM. PD-UBM, and PBS-treated mice lungs.

FIG. 8C illustrates neutrophil infiltration and lung injury in photomicrographs of lung sections from MRSA-infected FD-UBM, PD-UBM and PBS-treated mice lungs. Results are mean±SEM from three independent experiments; n=5 mice for each group. *p<0.05, **p<0.01, ***p<0.005, and ****p<0.001 for the comparison between groups.

FIGS. 9A-B graphically illustrate pre-formulated and un-digested UBM (U-UBM) protect host from acute severe respiratory MRSA infection.

FIG. 9A illustrates bacterial CFU in mouse BAL, lung, and total lung burden (BAL plus lung homogenate) in MRSA infected mice comparing treatment with PBS, U-UBM and PF-UBM.

FIG. 9B illustrates expression of inflammatory cytokines and chemokines including Cxcl1, Cxcl2, Cxcl3, IL-17, Tnf-α, and Nf-κb in MRSA infected mice comparing treatment with PBS, U-UBM and PF-UBM. Results are mean±SEM from three independent experiments; n=5 mice for each group. One-way analysis of variance (ANOVA) was used to compare drug-treated infected mice and PBS-treated infected animals, post hoc comparisons were made using the Dunnett's multiple comparison test when the P-value was significant (P<0.05). *p<0.05, **p<0.01, ***p<0.005, and ****p<0.001 for the comparison between groups.

### EXEMPLARY INVENTION

The invention described herein is directed to the use of ECMs such as UBM for the treatment of bacterial infections in humans and animals as exemplified by a murine pneumonia model of infection. By using the protocol described below, the antimicrobial activity of UBM in vitro and in vivo for host protection from MSSA-, MRSA-, Klebsiella pneumoniae and P. aeruginosa-induced infection was investigated. The results, described below in greater detail, show that UBM exhibited bactericidal activity toward a laboratory bacterial strain of MSSA and MRSA and exhibited appreciable anti-biofilm activity against multiple clinical MRSA isolates and P. aeruginosa.

Using a murine model of bacterial infection in humans, MSSA-, MRSA-, P. aeruginosa-, and K. pneumoniae-induced respiratory infections in mice result in significantly increased lung bacterial burden that is accompanied by increased recruitment of neutrophils and elevated pro-inflammatory cytokines and chemokines. Exogenous administration of UBM digest through intra-tracheal (i.t.) instillation protected the inoculated mice from severe lung pneumonia by significantly decreasing the bacterial burden and by attenuation of the bacterial cytokine/chemokine secretion. Furthermore, water reconstitution of pre-digested and lyophilized UBM that was kept at room temperature, as well as an un-digested particulate form of UBM, can similarly achieve the protected function of UBM against GPB- and GNB-induced pneumonia to provide an off-the-shelf and easily accessible resource to treat bacterial infection in humans and animals. These results of studies using the murine model of respiratory infection indicate that UBM is a viable alternative or supplement to conventional therapies for protection against bacterial infections in humans and animals, for example, respiratory MSSA, MRSA, and P. aeruginosa and K. pneumoniae bacterial infections.

Exemplary Materials and Methods UBM Digest Preparation

Articles for testing were prepared from a non-sterile form of micronized UBM powder (ACell, Inc., Columbia, Md.) labeled as undigested UBM (U-UBM) for in vivo testing as described below.

Briefly, proprietary ACell® UBM powder (MicroMatrix®) is manufactured by isolating the urinary bladder from a market weight pig, mechanically removing the tunica serosa, tunica muscularis externa, tunica submucosa, and tunica muscularis mucosa. The luminal urothelial cells of the tunica mucosa were dissociated from the basement membrane by washing with deionized water. The remaining tissue consisted of epithelial basement membrane, and subjacent lamina propria of the tunica mucosa which is referred to as UBM. The remaining tissue is next decellularized by agitation in 0.1% peracetic acid with 4% ethanol for 2 hours at 150 rpm. The tissue was then extensively rinsed with 1×PBS and sterile water. No cross-linking agents, detergents, peptidases or proteases were used in the preparation of UBM. Subsequently, the tissue was lyophilized and then milled into a powder particulate form using a Wiley Mill (Thomas Scientific, N.J.) with a #60 mesh screen. The UBM powder was then sifted through a 150-micron screen using a Tapping Sieve Shaker (Gilson, Ohio) for four hours. Alternatively, lyophilized UBM was cut to small piece to fit a Cryomill sample chamber and was processed using a Cryomill instrument (Retsch, Haan, Germany) for two and a half hours by alternating cooling, shaking and resting steps In an alternative embodiment, micronized UBM powder was also enzymatically digested to create a stock UBM digest solution as previously described in D. O. Freytes, J. Martin, S. S. Velankar, A. S. Lee, S. F. Badylak, Preparation and rheological characterization of a gel form of the porcine urinary bladder matrix, Biomaterials 29(11) (2008) 1630-7, incorporated by reference in its entirety herein. Briefly, a solution of 0.01 HCl and 120 mg of porcine pepsin (Sigma Aldrich, St. Louis, Mo.) was mixed until dissolved. 1.2 g of non-sterile UBM (MicroMatrix®) particulate made according to T. W. Gilbert, D. B. Stolz, F. Biancaniello, A. Simmons-Byrd, S. F. Badylak, Production and characterization of ECM powder: implications for tissue engineering applications, Biomaterials 26(12) (2005) 1431-5, incorporated by reference in its entirety herein, was added to the pepsin solution to achieve the desired stock solution concentration and stirred at room temperature until fully dissolved, approximately 48 hours. The digested UBM solution was then cooled to 5° C. using an ice bath. While stirring, 12 ml of 10X phosphate buffered saline (PBS), 5 mL 0.02M NaOH, and 3 ml deionized water were added to neutralize the UBM digest. The pH was then tested to ensure neutralization was achieved. For the pre-formulated UBM (PF-UBM), the resulting neutralized digest was aliquoted in centrifuge tubes and frozen overnight. The tubes of neutralized PF-UBM digest were then removed and lyophilized, and the samples were then packaged and sterilized using electron beam irradiation. The samples were stored at room temperature until needed for experiments. For both freshly digested UBM (FD-UBM) and the PF-UBM groups (pre-formulated, lyophilized and sterilized digest), test articles were ultimately prepared at the desired final concentrations for individual experiments as described below.

Mice and Animal Husbandry

Wild-type FVB/NJ mice were purchased from Jackson Laboratory (Bar Harbor, Me.) and maintained in a specific pathogen-free status in a 12-h light/dark cycle. All procedures were conducted using mice 8-9 weeks of age maintained in ventilated micro-isolator cages housed in an American Association for Accreditation of Laboratory Animal Care (AAALAC)-accredited animal facility. Protocols and studies involving animals were conducted in accordance with National Institutes of Health guidelines and approved by the Institutional Animal Care and Use Committee at the University of Pittsburgh.

Bacteria

The gram-positive (GPB) Staphylococcus aureus strains (MSSA ATCC #49775 and MRSA USA300), and gram-negative GNB Pseudomonas aeruginosa (PA01, ATCC BAA-47) and Klebsiella pneumoniae (KP, B3) were used for all experiments. These gram-positive and gram negative strains of bacteria are known to have an impact on human health. Bacterium obtained from a single colony was stored in aliquots at −80° C. in 15% glycerol/tryptic soy broth (TSB). For each experiment, an aliquot of bacteria was grown for 16 h at 37° C. in autoclaved TSB with shaking. An aliquot of the overnight grown bacteria was then diluted 1 ml into 5 ml fresh TSB and incubated for an additional 2 h at 37° C. with shaking. Bacteria were washed twice and resuspended in 10 ml phosphate-buffered saline (PBS).

Pulmonary Toxicity

In vivo pulmonary toxicity of UBM was examined by intra-tracheal (i.t.) administration into mouse lung. FVB/NJ mice were lavaged i.t. with 50 μl PBS at different concentrations of UBM per ml, ranging from 1 mg/kg to 10 mg/kg. Lung tissues were lavaged as described in Y. P. Di, Assessment of pathological and physiological changes in mouse lung through bronchoalveolar lavage, Methods Mol. Biol. 1105 (2014) 33-42, incorporated by reference in its entirety herein, harvested at 24 hours after UBM administration, and analyzed for toxicity by total protein, lactic acid dehydrogenase (LDH), total leukocytes, and differential cell counts in bronchoalveolar lavage (BAL) as well as by gene expression using real-time PCR analysis.

In vivo Exposure of Mice to Bacteria

Mice were anesthetized with inhalation of isoflurane and treated with ATCC#49774, USA300, or PA01 through intranasal (i.n.) instillation of ˜2×10⁶ CFU (regular infection) or ˜2×10⁷ CFU (severe infection) per mouse in 50 μl PBS. Control mice were intranasally inoculated with 50 μ1 of PBS. One hour after bacterial inoculation, mice were intra-tracheally instilled with 50 μl of UBM at 10 mg/kg and control mice with 50 μl of PBS. Mice were then sacrificed 14 hours after UBM administration to investigate the acute host response to bacterial infection and subsequent treatment.

CFU Assay

The number of CFU was determined by serial dilution and quantitative culture on TSB agar plates. The left lung lobe was homogenized in 1 ml saline and placed on ice. Dilution of 100 μl of lung tissue homogenate or bronchoalveolar lavage fluid (BALF) was mixed with 900 μl saline. Four serial 10-fold dilutions in saline were prepared and plated on TSB agar plates and incubated for 18 h at 37° C., each dilution plated in triplicate. The colonies were then counted and surviving bacteria were expressed in log₁₀ units.

BALF and Cell Differential Counts

At 15 h after treatment of bacterial infection (14 h after UBM administration), mice (5 mice/group) were anesthetized with 2.5% tribromoethanol (Avertin). The trachea was cannulated, the lungs were lavaged twice using 1 ml saline, and the BALF samples pooled. A 16 μl aliquot was stained with 4 μl Acridine orange (MP Biomedical, Santa Ana, Calif.), and cells were counted with a Vision Cell Analyzer cell counter (Nexcelom, Lawrence, Mass.). An additional aliquot was placed onto glass microscope slides (Shanon Cytospin; Thermo Fisher, Pittsburgh, Pa.), stained with Diff-Quick; cell differential was determined microscopically. A total of 400 cells of every slide were counted at least twice for inflammatory cell differential counts.

Real-Time PCR Analysis

Total mRNA was isolated from the upper two lobes of right lung tissues of WT and Spluncl KO mice using Trizol reagent (Life Technologies, Carlsbad, Calif.). Quantitative PCR (qPCR) was performed using ABI7900HT (Applied Biosystems, Foster City, Calif.) and primers of Muc5ac, Muc5b, CCSP, Foxj1, Cxcl1, Cxcl2, Cxcl5, NF-κB, IL-6, IL-10, IL-1a, Ccl20. Validation tests were performed to confirm equivalent PCR efficiencies for the target genes. Test and calibrator lung RNAs were reverse transcribed using a High-Capacity cDNA reverse transcription kit (Life Technologies), and PCR was amplified as follows: 50° C. for 2 min, 95° C. for 10 min, 40 cycles; 95° C. for 15 s; 60° C. for 1 min. Three replicates were used to calculate the average cycle threshold for the transcript of interest and for a transcript for normalization (β-glucuronidase [GUS-B]; Assays on Demand; Applied Biosystems). Relative mRNA abundance was calculated using the AA cycle threshold (Ct) method.

Cytokine Assay

Cytokine levels in BAL were quantified using the mouse Cytokine Multiplex Panel Milliplex assay (Millipore, Billerica, Mass.). The expressions of IL-10, IL-6, IL-10, IL-12(p70), IL-17, IFN-γ, TNF-α, GM-CSF, KC, IP-10, VEGF and MIP-1α were analyzed using the Luminex assay system, based on manufacturer's instructions and as previously described in Y. Zhang, R. Birru, Y. P. Di, Analysis of clinical and biological samples using microsphere-based multiplexing Luminex system, Methods Mol Biol 1105 (2014) 43-57. Standard recombinant protein solution was used to generate a standard curve for each analyzed protein. Absolute cytokine concentrations were calculated from the standard curve for each cytokine.

Lung Histopathology

Lung tissues were harvested at 15 h after infection, inflation fixed in situ with 4% paraformaldehyde at 10 cm H₂O for 10 minutes with the chest cavity open. The right lobe was embedded in paraffin and 5 μm sections were prepared. Sections were stained with hematoxylin and eosin, and histological evaluation was performed to examine bacterial infection-induced pathological severity. The stained lung sections were evaluated in a double-blind fashion under a light microscope, using a histopathologic inflammatory scoring system.

Biofilm Assay

A slightly modified version of the microtiter plate assay developed by O'Toole and Kolter was used as described in Y. Liu, M. E. Di, H. W. Chu, X. Liu, L. Wang, S. Wenzel, Y. P. Di, Increased susceptibility to pulmonary Pseudomonas infection in Splunc 1 knockout mice, J Immunol 191(8) (2013) 4259-68 and G. A. O'Toole, R. Kolter, Flagellar and twitching motility are necessary for Pseudomonas aeruginosa biofilm development, Molecular microbiology 30(2) (1998) 295-304, both incorporated by reference in their entirety herein.

Briefly, overnight planktonic cultures of bacteria were inoculated into 100 μL of DMEM in a 96-well culture-treated polystyrene microtiter plate (Fisher Scientific, Pittsburgh, Pa.) with or without UBM or antibiotic controls. Wells filled with growth medium alone were included as negative controls. After 3 hour incubation at 37° C., surface-adherent biofilm formation was measured by staining bound cells for 15 minutes with a 0.5% (w/v) aqueous solution of crystal violet. After rinsing with distilled water, the bound dye was released from the stained cells using 95% ethanol, and optical density was determined at 590 nm.

Data Analysis

Data are expressed as mean±SEM. Statistical comparisons between the groups of mice were made using ANOVA, followed by Dunnett's multiple comparison test (one way ANOVA). A p value<0.05 was considered to be statistically significant.

Results

In Vitro Studies UBM displays in vitro antibacterial activity

To determine if UBM contains any component that may display growth inhibition on bacteria, we suspended a micronized UBM powder in saline at a concentration of 4 mg/ml (ACell, Inc.) to test its antimicrobial activity. A panel of multiple common respiratory bacterial infections including GPB (MMSA and MRSA) as well as GNB (Pseudomonas aeruginosa and Klebsiella pneumoniae) were tested because they are the most prevalent bacterial strains that are frequently associated with respiratory infections.

Two different preparations of UBM were carried out. The first was to simply suspend the powder form of UBM (MicroMatrix®, ACell, Inc.) in PBS, centrifuge down the undissolved materials, and collect the soluble part of the UBM (UBM supernatant) with the notion that antimicrobial agents such as antimicrobial peptides (AMPs) would remain active in the supernatant in inhibiting bacterial growth.

The second method was to enzymatically digest the UBM with pepsin as described above to extract all potential antimicrobial molecules such as peptides from the matrix materials (digested UBM). All tested bacteria grown at log phase were used to determine the antimicrobial activity of non-digested and digested UBM materials in direct killing of bacteria.

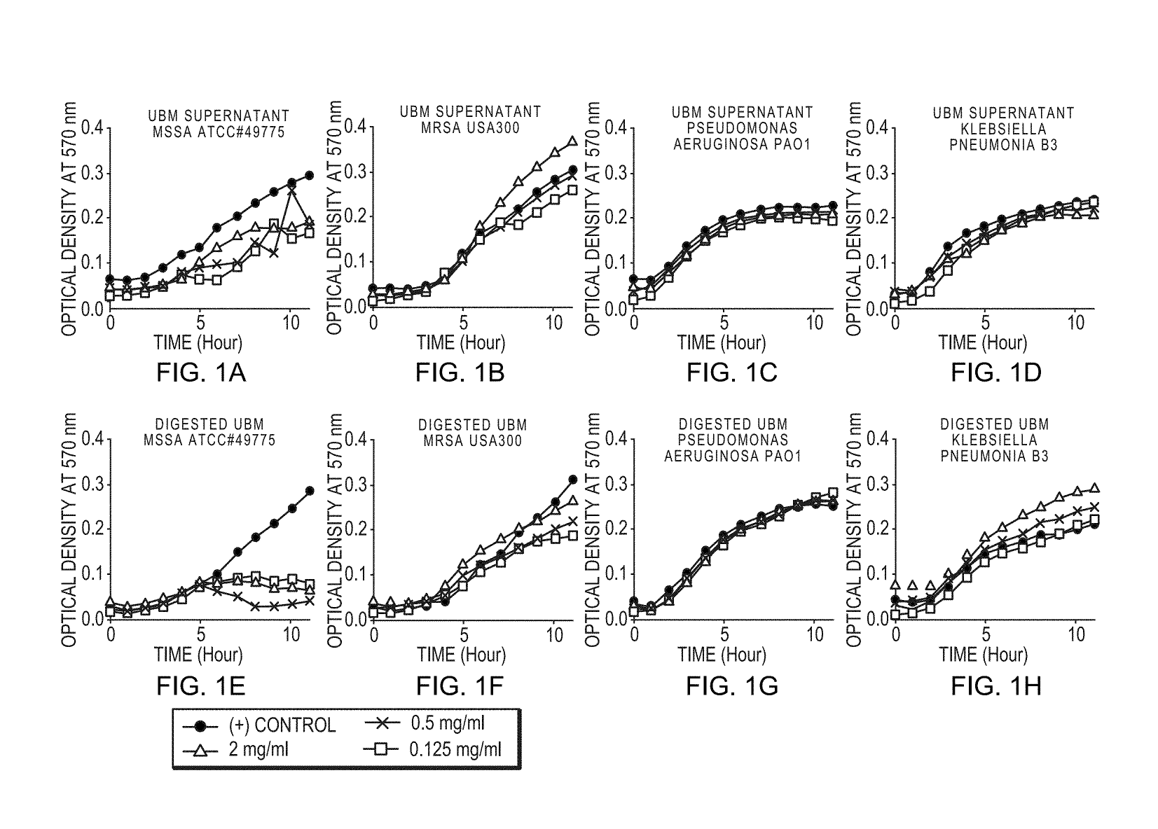

Referring to FIGS. 1A-C, the UBM supernatant did not display any noticeable antimicrobial activity against GPB (MSSA and MRSA) (FIGS. 1A, 1B) or GNB (PA and KP) (FIG. 1C, 1D). The digested UBM has bactericidal activity in vitro against MSSA (FIG. 1E) but not in vitro against other GPB (MRSA; FIG. 1F) or GNB (PA and KP; FIGS. 1G, 1H). It appears that some antimicrobial molecules are released from the matrix after protease digestion instead of just the PBS-soluble component that helped UBM-based bactericidal activity because the digested UBM displayed enhanced antibacterial activity compared to the soluble component of UBM (FIG. 1). Therefore, the digested form of UBM was used for two in vivo experimental groups within this study described below. In another experimental group, micronized undigested UBM powder in vivo was used as a lavage in the murine pneumonia model, based upon the expectation that the material would be degraded upon instillation into the lungs.

In Vivo Studies-Tissue Tolerance to UBM UBM is well-tolerated in the lung and does not display pulmonary toxicity

The following studies demonstrate that UBM is not toxic to the lung and does not cause lung injury.

Eight to nine week old FVB/NJ mice were intra-tracheally (i.t.) instilled into mouse lung with 50 μl digested FD-UBM at different concentrations (0.1, 0.5, 1, and 2 mg/ml) resulting in an administered dosage of 0.25, 1.25, 2.5, or 5 mg/kg). No significant changes were identified when comparing multiple indicators of toxicity (including total cell number and LDH in BAL, gene expression of lung epithelial cells and Nf-κb) between UBM instilled mouse groups and control group of mice that received only the vehicle control. Higher concentrations of the digested UBM (4 mg/ml) for a resulting dosage of 10 mg/kg in mouse lung (200 μg/mouse lung) were also evaluated.

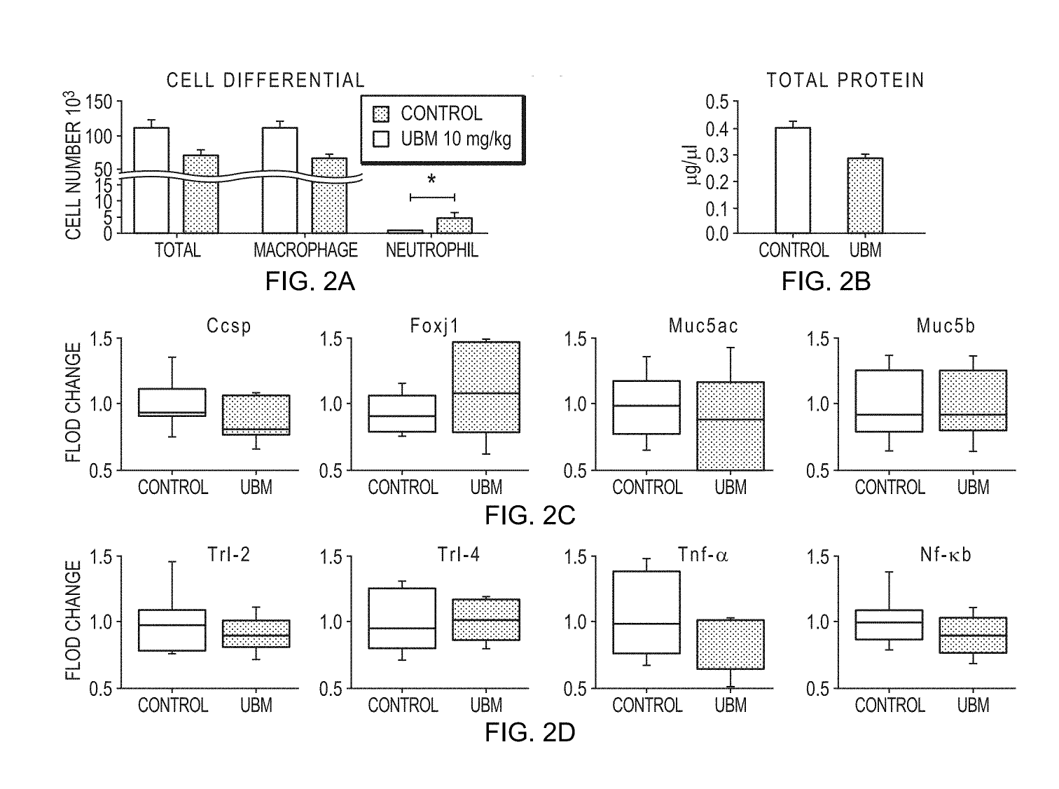

Referring to FIG. 2, even at the higher UBM concentration of 10 mg/kg, in nearly all measurements remained comparable in mice between the vehicle and FD-UBM treated groups. As shown in FIG. 2A, a minimal increase of neutrophils was observed in the FD-UBM-treated group, which accounts for about 3-4% of the total leukocytes in the mouse lung, but was not statistically significant. Similarly, the total protein in the lungs (as an indicator for lung injury) shown in FIG. 2B, did not show a difference between the PBS control and FD-UBM treated mouse groups. Referring to FIG. 2C, after the administration of UBM into mouse lung (10 mg/kg for a total of 200 μg /mouse), the expression of epithelial cell related genes including Ccsp (for Club cells), Foxj1 (for ciliated cells), and Muc5ac (for Goblet cells), and Muc5b (for mucous cells) did not show any noticeable changes, nor did the expression of inflammation associated genes in TLR-2, TLR-4, Tnf-α, and Nf-κb as shown in FIG. 2D. These data suggest that administration of UBM into mouse lung at the highest concentration (10 mg/kg) did not disturb lung epithelial cell integrity or elicit an inflammatory response.

In Vivo UBM Antimicrobial Studies UBM displays in vivo antimicrobial activity against MSSA in a murine model of respiratory infection.

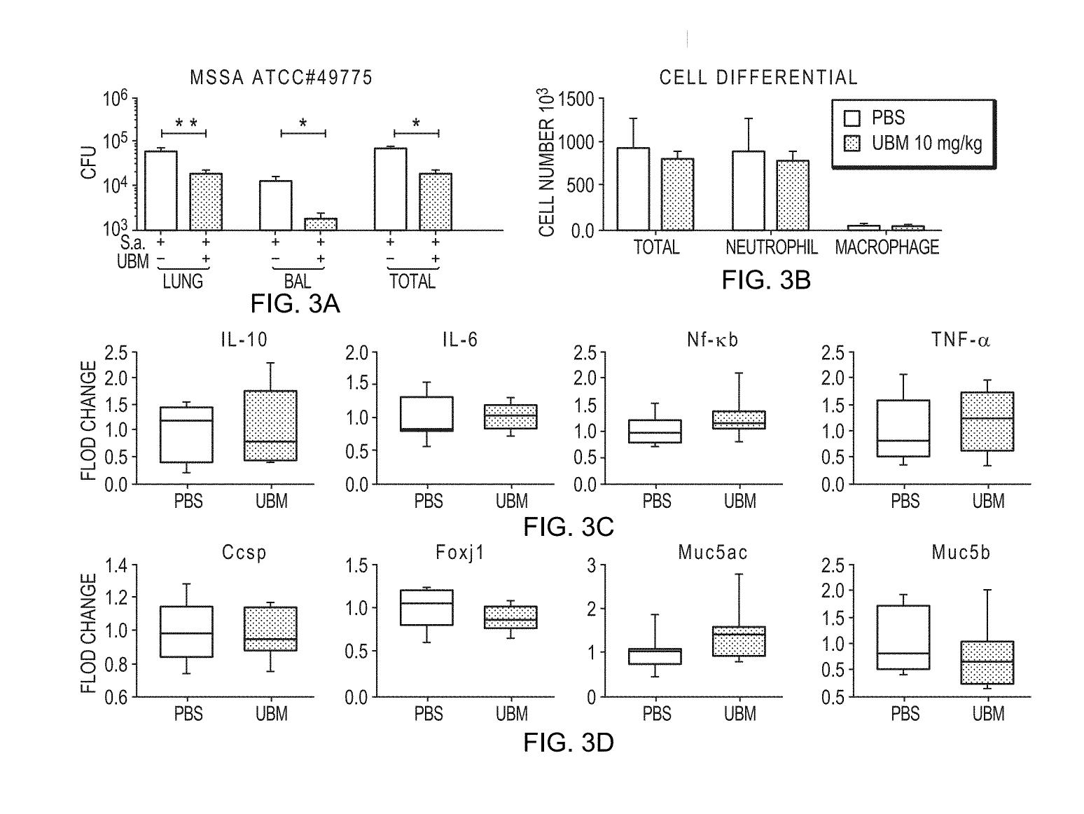

To test if exogenous administration of UBM is capable of protecting host from S. aureus-induced infection, a murine pneumonia model was used to determine UBM-based antimicrobial activity in vivo. Age-matched FVB/N mice were intratracheally (i.t.) instilled with MSSA (ATCC #49775) at a dose of ˜2×10⁶ CFU/Lung. FD-UBM 50 μl at 10 mg/kg was delivered (i.t.) at 1 hour after the bacterial infection to test the therapeutic effects of UBM on respiratory bacterial infection. At 15 hours after bacterial infection, illustrated in FIG. 3A, mice treated with FD-UBM showed significantly decreased bacterial numbers in both BAL and lung. Thus, the total lung bacterial burden in mouse groups treated with UBM at one hour after bacterial infection was significantly decreased by more than six folds compared to the initial lung bacterial burden. Unexpectedly, shown in FIG. 3B, the difference in bacterial burden did not affect the total number of leukocytes, as both PBS- and FD-UBM-treated groups of mice showed no statistical difference of total inflammatory cell counts and differential cell counts of macrophages and neutrophils in BAL. There was also no significant difference in the expression of anti-inflammatory cytokine IL-10 and pro-inflammatory cytokine IL-6, Nf-κb and Tnf-α illustrated in FIG. 3C and no noticeable changes were observed in airway epithelial cell related genes shown in FIG. 3D.

UBM Effectively Protects Mice From MRSA-Induced Respiratory Infection

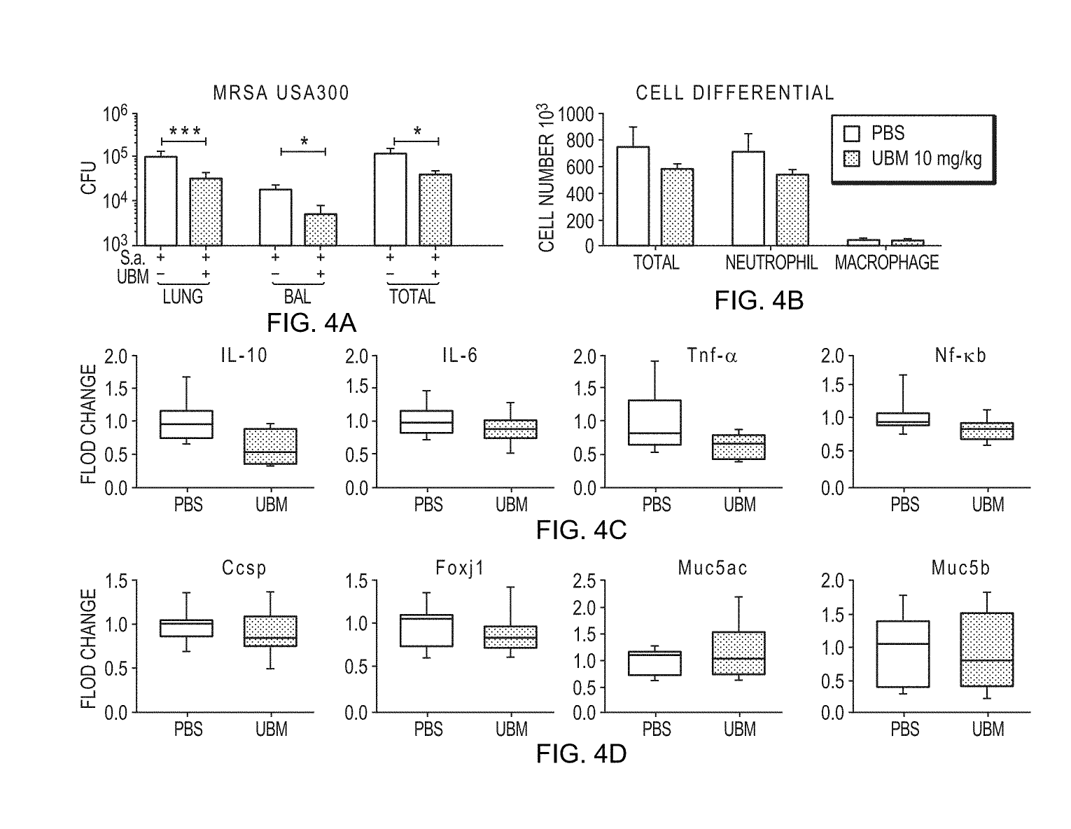

A similar set of murine Staphylococcus aureus infection experiments to those described above using MSSA were carried out using MRSA (USA300) in the murine pneumonia model. Referring to FIG. 4A, the FD-UBM MRSA-infected mice had significantly increased bacterial numbers in both the BAL and lung compared to studies using MSSA described above. However, the majority of the bacteria (MRSA) were bound to lung tighter than MSSA and remained in the lung (˜10⁵ CFU/lung, ˜84% of total lung bacterial CFU) rather than being rinsed out in the BAL (˜1.8×10⁴ CFU/lung).

Advantageously, the exogenously administered UBM appeared to be effective against MRSA in vivo, as this treatment displayed antimicrobial activity in mice against MRSA-induced respiratory infection. Greater than an 80% reduction of total lung MRSA bacterial burden was observed in mice treated with FD-UBM, as opposed to mice treated with only a PBS control. The total leukocytes in FD-UBM-treated BAL from MRSA exposed mice were slightly less than PBS control group but did not yield statistical significance (FIG. 4B). Illustrated in FIG. 4C and 4D, the inflammation-related and epithelial cell-associated gene expression of UBM-treated, MRSA exposed mice showed trends to display lower expression than non-UBM treated MRSA exposed mice but did not yield statistical significance.

UBM Bioactivity Prevents Bacterial Attachment In Vivo

UBM-mediated antimicrobial mechanism that is common to both MSSA and MRSA does not appear to have a direct killing activity against MRSA in vitro (FIG. 1), but still displays excellent in vivo antimicrobial activity against MRSA (FIG. 4). Since inoculated bacteria must attach to the epithelium to avoid being pushed out of lung by muco-ciliary clearance in the murine pneumonia model, UBM administration into mouse lung evidently prevents the bacterial attachment to mouse lung epithelium.

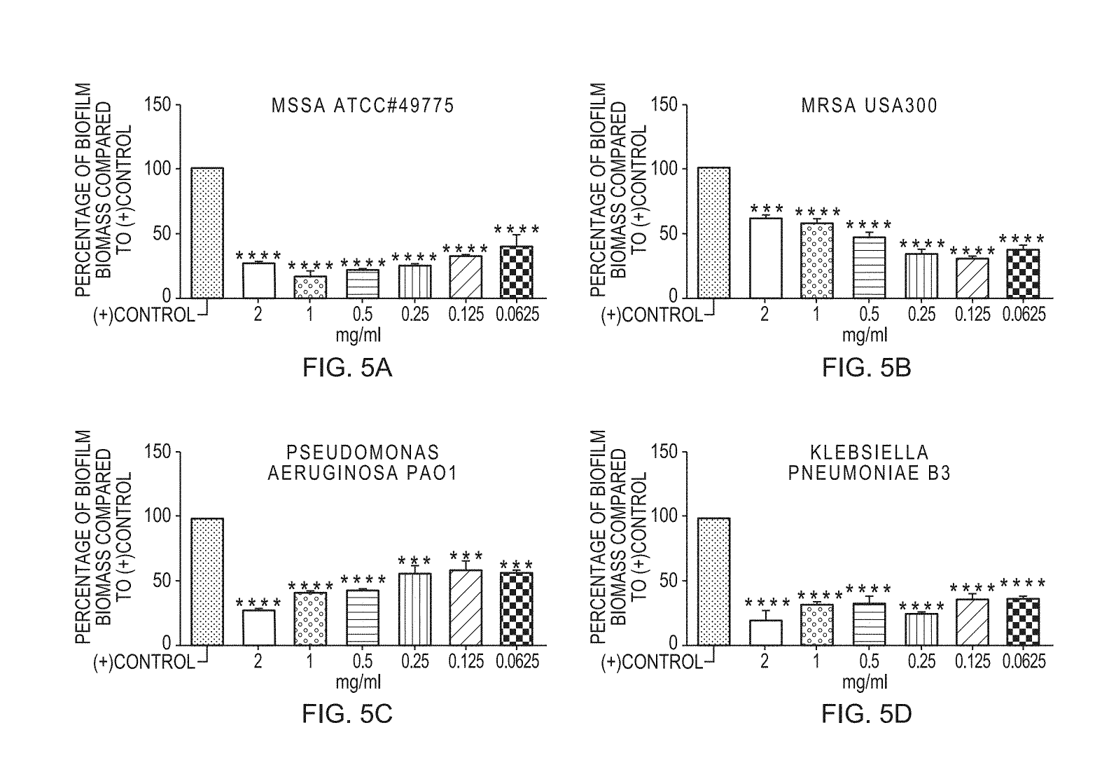

Bacterial attachment of MSSA and MRSA in the presence of FD-UBM (described below) was investigated at various concentrations through the use of a biofilm formation assay. Determination of anti-biofilm effects of FD-UBM on MSSA MRSA, PA and KP was carried out by measuring the biofilm biomass on abiotic surfaces via crystal violet staining (OD620) as described above. FD-UBM at concentrations higher than 0.0625 mg/ml effectively decreased the bacterial attachment of MSSA, shown in FIG. 5A and MRSA shown in FIG. 5B to the culture plate, and thus prevented the initiation of biofilm formation.

To determine if the FD-UBM-mediated anti-biofilm activity was broad spectrum or limited to just GPB, the anti-biofilm activity of FD-UBM was tested in the aforementioned biofilm formation assay against the relevant respiratory GNB pathogens including P. aeruginosa (PA) and K. pneumoniae (KP). Our results indicated that FD-UBM also possesses excellent anti-biofilm activity against GNB (FIGS. 5C and 5D).

UBM Also Protects Mice From Pseudomonas aeruginosa-induced Respiratory Infection

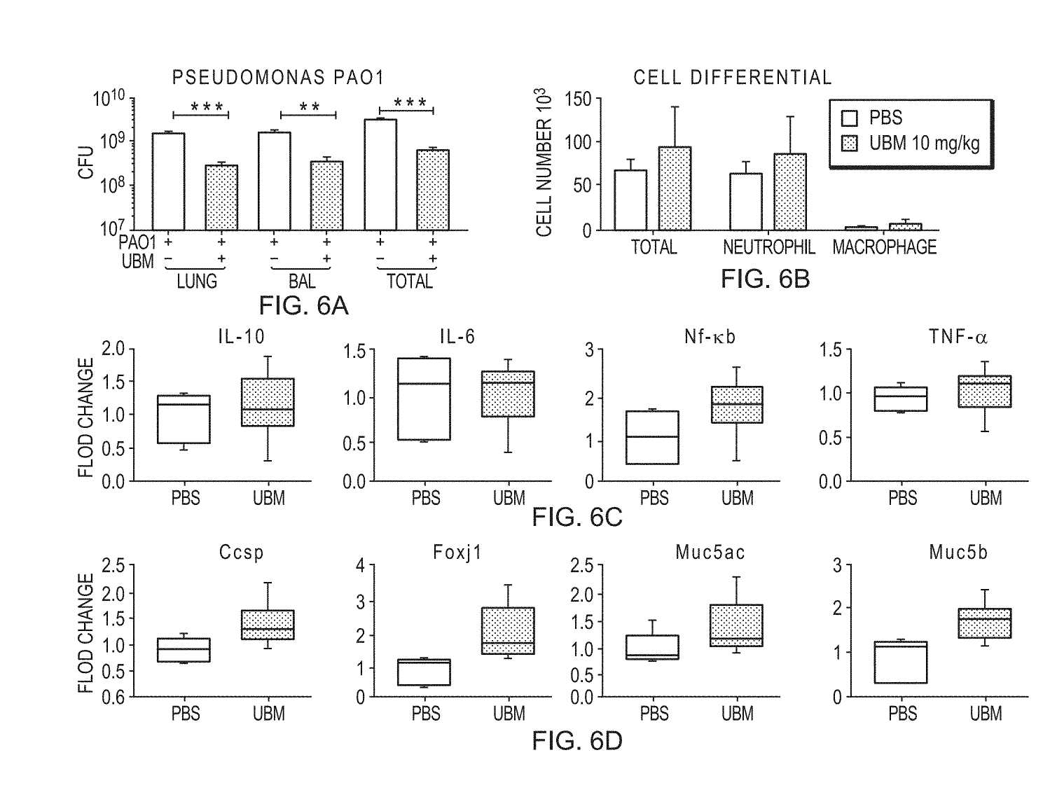

To further evaluate if the UBM-mediated anti-biofilm activity could also protect host from GNB bacterial infection, murine respiratory infection experiments were similarly carried out using P. aeruginosa (PAO1). Age-matched wild-type FVB/NJ mice were intra-tracheally inoculated with ×10⁷ CFU P. aeruginosa (PAO1) per mouse. The exogenously administered pre-formulated UBM (PF-UBM) also effectively protected mice against GNB P. aeruginosa-induced respiratory infection (FIG. 6A-D). These data suggest that the PF-UBM-mediated anti-biofilm activity, demonstrated in FIG. 5, likely contributes to the common protective mechanisms for the host to fight bacterial infection in vivo.

Pre-Formulated UBM Maintains Antimicrobial Activity After Reconstitution

Freshly digested UBM (FD-UBM) was used in the in vitro studies (FIGS. 1 and 5) since intact UBM is known not to degrade in vitro, and was used in vivo for ease of comparison. However, the use of freshly digested UBM is not practical in the clinical setting. Due to the need for a rapid response to injury in a lung infection, an off-the-shelf form of pre-formulated lyophilized and sterilized UBM (PF-UBM) digest to maintain the characteristics of the freshly digested UBM for lung protection is advantageous over freshly digested UBM.

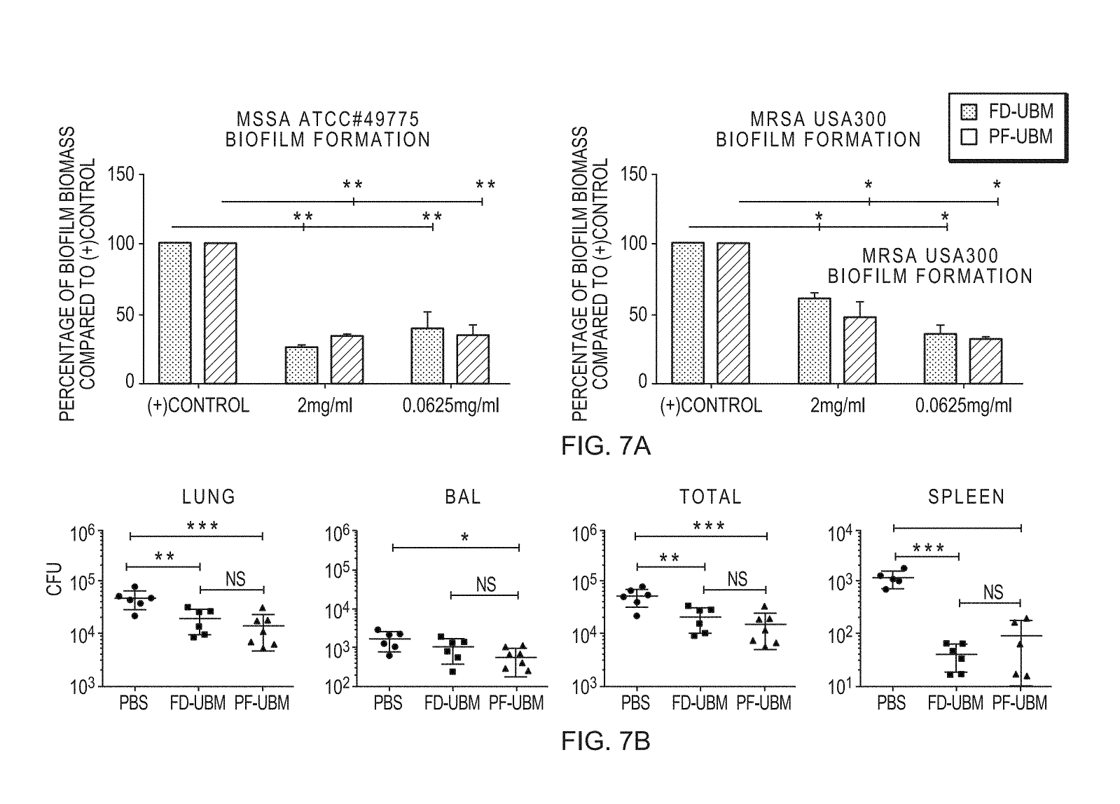

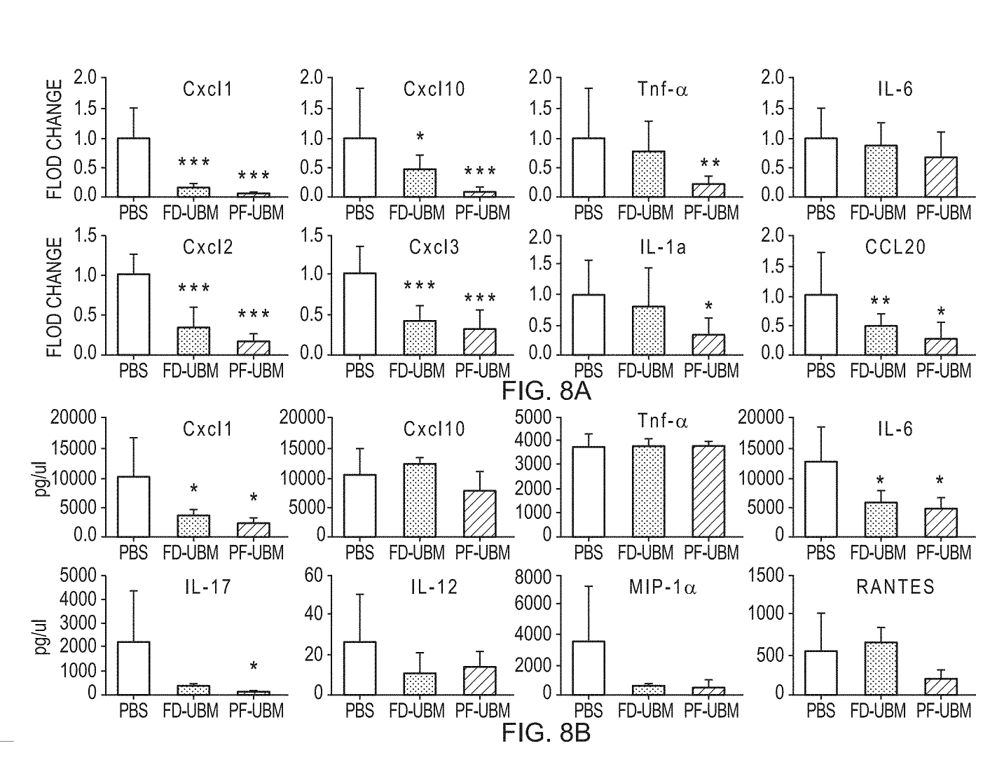

For these studies, three batches of lyophilized PF-UBM were separately tested for their in vitro and in vivo antimicrobial activity and compared with FD-UBM (made in the laboratory immediately before use) using the anti-biofilm measurement method described above. The PF-UBM solution, which may be stored for many years, showed very similar in vitro inhibition of P. aeruginosa and MRSA to the FD-UBM (FIG. 7A). The lyophilized PF-UBM also demonstrated similar in vivo antimicrobial activity as PF-UBM in protecting host from P. aeruginosa and MRSA in murine pneumonia infection models (FIG. 7B).

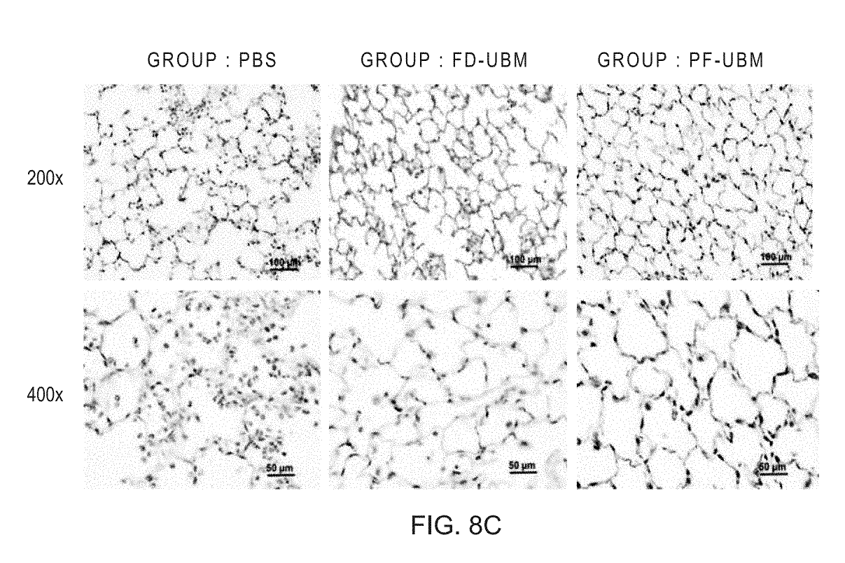

To further evaluate the effects of PF-UBM and FD-UBM treatments on the gene and protein expression of inflammatory response-related cytokines and chemokines, real time qPCR and Luminex were used to analyze mouse lung and BAL samples, respectively, as shown in FIG. 8A. Mice were infected with approximately 2×10⁶ CFU of MRSA i.t. and treated with 10 mg/kg of either PF-UBM or FD-UBM i.t. one hour after inoculation with MRSA. Since several genes, as examined in FIGS. 3 and 4, did not show difference between PBS- and UBM-treated groups of mice, additional genes and proteins were selected for evaluation. Unexpectedly, referring to FIG. 8A, noticeably lower gene expression was detected in FD-UBM treated mice than PBS treated control mice with regards to Cxcl1, Cxcl2, Cxcl3, Cxcl10, and Ccl20 but not Tnf-α, IL-1α, and IL-6. Additionally, PF-UBM demonstrated significant inhibition on all examined gene expression of Cxcl1, Cxcl2, Cxcl3, Cxcl10, Cc120, Tnf-α, and IL-1α except IL-6 (FIG. 8A). The secreted protein amount in BAL of Cxcl1 and IL-6 was significantly lower in both PF-UBM and FD-UBM treated mice than PBS-treated control mice (FIG. 8B). There was no significant difference regarding the secretion of Cxcl10, IL-12, Tnf-α, and RANTES in BAL while IL-17 and MIP-1α showed trends of low expression after UBM treatment (FIG. 8B).

The decreased expression of inflammatory cytokines and chemokines was also reflected in lung pathological analyses of MRSA (USA 300) infected mice after UBM treatment illustrated in FIG. 8C. Both PF-UBM and FD-UBM treated mice also displayed enhanced bacterial clearance against MRSA (FIG. 8C). The results indicate that both PF-UBM and FD-UBM are comparable and effective in protecting host from MRSA induced respiratory infection.

Pre-Formulated and Undigested UBM Express A Protective Effect Against High Doses of Bacteria Induced Respiratory Infection

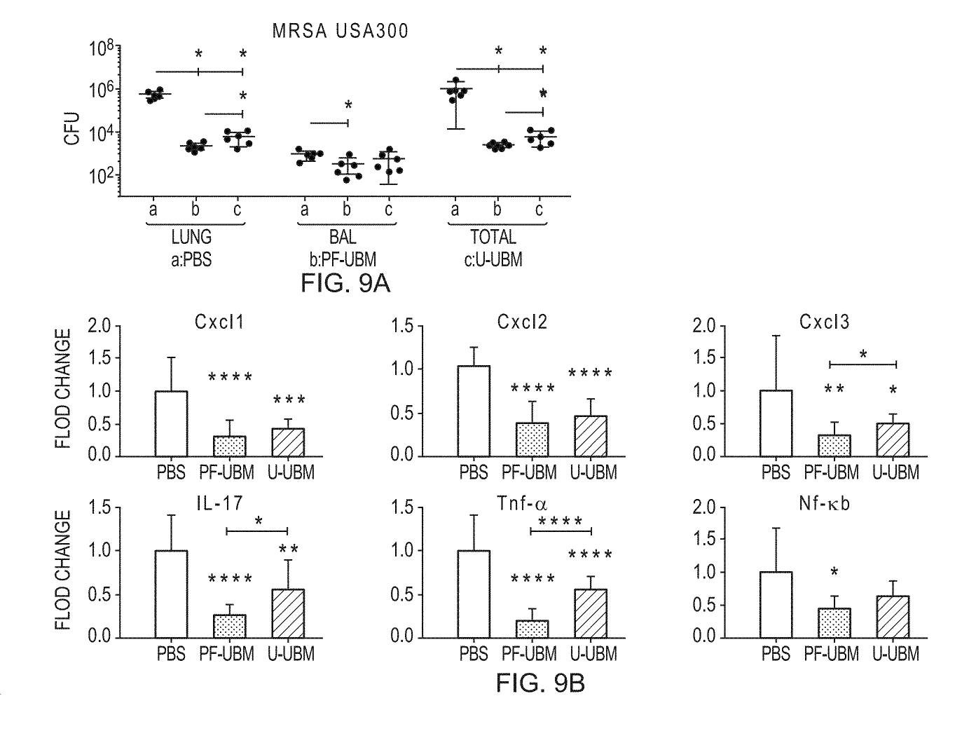

To test the utility of UBM in treating acute severe GPB and GNB-induced respiratory infections of patients, MRSA and P. aeruginosa were inoculated with a higher bacterial burden (10×) than previously used CFU in the murine pneumonia model. MRSA (USA300) on P. aeruginosa was instilled through i.n. into FVB/N mice at a dose of ˜2×10⁷ CFU/Lung. PF-UBM and an undigested, intact form of particulate UBM (U-UBM) suspended in saline at 10 mg/kg were delivered (i.t.) at 1 hour after the bacterial infection. Referring to FIG. 9, both PF-UBM and U-UBM treatments significantly decreased total lung bacterial burden compared to the PBS-treated mice group.

Conclusions

The results in the series of in vitro and in vivo experiments conducted to evaluate the potential antimicrobial benefits of using UBM as an exemplary ECM in a therapeutic application to fight GPB and GNB-induced bacterial infection in patients described herein indicate that a digested form of UBM displays better antimicrobial activity than the supernatant of physiologic buffer PBS-extracted UBM against MSSA in vitro. Although digested UBM did not show direct bactericidal activity against MRSA or P. aeruginosa in vitro, intra-tracheal instillations of PF-UBM and U-UBM, effectively protected against both MSSA-, MRSA-, and P. aeruginosa infected mice in murine respiratory pneumonia models. Since S. aureus and P. aeruginosa are common pathogens associated with infection, antimicrobial activity of UBM against these infections is relevant, not only to the frequent use of UBM to treat a variety of wounds, including traumatic acute injuries and burns in many tissues including but not limited to skin and lung, but potentially as a non-topical therapeutic application, e.g., inhalation or systemic therapeutic application.

The in vivo antimicrobial activity of undigested UBM, freshly digested UBM, and preformulated digested UBM in protecting the host from bacterial-induced pneumonia averaged an approximate 5-6 fold decrease (˜80% to 85% protection) in total lung bacterial burden. The demonstrated in vivo results illustrate the advantages of UBM in reducing bioburden since other inflammation-related gene knockout mice (such as IL-17 knockout) used in other studies were only able to reduce the MRSA bacterial burden in the lung by about 2-3 fold. Furthermore, the pre-formulated PF-UBM was effective at reducing MRSA infection even when a severe inoculation (10-times higher CFU of MRSA than normal) was administered into mice lungs to induce severe respiratory MRSA infection as demonstrated in FIG. 9. The increased lung bacterial burden in PBS-treated mice was more than 250-fold higher than PF-UBM-treated mice and 87-fold higher than U-UBM-treated mice. These results show that UBM is therapeutic in vivo in the bacterial infection setting in mammals. Not to be bound by theory, it is believed that UBM may permit only a limited number of bacteria to attach to epithelium while UBM prevents MRSA from homing to the mouse lung.

One of the likely mechanisms by which UBM exhibits strong antimicrobial activity in vivo is its strong anti-biofilm formation activity after in vivo enzymatic degradation. Bacteria tend to group together and stick to each other on a surface to form biofilms and subsequently undergo changes in phenotype and gene expression. It is estimated that more than 80% of human infectious diseases are directly related to bacterial biofilm formation, but the majority of bacterial research to date has been performed on free swimming, planktonic bacteria and not biofilm-associated bacteria. Biofilm-associated bacteria are much more critical than planktonic forms in the pathogenesis of bacterial colonization. One of the potential modes of UBM on biofilm formation is due to the biophysical property of UBM which may slow down bacterial homing to the lung and/or form a protective layer on the epithelium and result in decreased biofilm formation on epithelial surfaces. Components of UBM may interact or neutralize the ability of bacteria to attach to lung epithelial cells.

The results described herein illustrate that exogenously administered UBM in vivo provides an efficient protection against bacterial infections. The enhanced bacterial clearance observed in UBM-treated mice may occur due to the interaction of UBM with other antimicrobial peptides such as defensins and/or antimicrobial proteins such as lysozyme to potentiate its antibacterial activities.

Cytokines also play an important role in regulation and modulation of immunological and inflammatory processes. Normally, following the recognition of microbial products, TLR-mediated signaling within epithelial cells results in the production of TNF-α and IL-1β, two early-responsive cytokines that regulate subsequent recruitment of neutrophils. A well-regulated and balanced production of inflammatory mediators is critical to an effective local and systemic host defense against bacterial infection.

In the studies disclosed herein, most of the inflammatory cytokines such as IL-6, IL-10, and TNF-α did not change noticeably between PBS- and UBM-treated mice after a common dosage-induced bacterial infection (FIGS. 3, 4 and 5). However, several cytokines and chemokines, were significantly decreased in UBM treated mice groups compared with PBS-treated mice group (FIGS. 8 and 9).

One of the important and unexpected advantages of UBM identified in this study over known methods of treatment of bacterial infection is that the pre-formulated (pre-digested, lyophilized, and sterilized) PF-UBM retains its antimicrobial activity against MSSA and MRSA-induced infection even after prolonged storage at room temperature. The PF-UBM used in this study was sterilized and stored at room temperature conditions for up to 6 months prior to use in both in vitro and in vivo experiments. The PF-UBM with prolonged stability can be stored for years at room temperature as an off-the-shelf product, further enhancing its utility as an easily accessible antimicrobial agent that can be used to treat microbial infection.

Another advantage identified in these studies is that undigested U-UBM also exhibited excellent antimicrobial activity against MRSA-induced respiratory infection. Again, not to be bound by theory, a potential mechanism is that U-UBM is digested by secreted proteases in the host airway, thus resulting in the in situ digestion and breakdown of undigested UBM to protect host from bacterial infection, similar to the observed anti-microbial effects of digested PF-UBM and FD-UBM. Preparation of the ECM-derived compositions described above, such as but not-limited to UBM, formulated in the absence of protein cross-linkers, may be advantageous for use of the compositions in treatment of bacterial infections, including but not limited to respiratory infections. In situ breakdown of cross-linked proteins may exceed the capacity of host proteases and peptidases.

In summary, the inventions disclosed herein include but are not limited to the use of the broad spectrum antibacterial activity of UBM against bacterial pathogens using in vivo approaches within airways. Additionally, UBM may be used, for example, as a treatment for or to improve resistance to S. aureus and P. aeruginosa, studied here as exemplary bacterial infections, and other bacterial infections in wounds, burns, persistent infections of the skin, comminuted bone fractures, cystitis, cellulitis, nosocomial infections, and airway and other tissue infections. As non-limiting examples, UBM may be useful for therapy of early life bacterial colonization in cystic fibrosis patients. UBM-mediated antimicrobial activity is an alternative approach to efficiently combat bacterial infections such as bacterial infection of airways in immune-competent and immune-compromised patients.

We claim:

1. A method for the treatment of a respiratory infection in a patient, comprising:
administering to the patient via an airway an effective dose of a non-cross-linked, micronized powder obtained from a devitalized native extracellular matrix material and processed at room temperature, said devitalized native extracellular matrix (ECM) selected from the group consisting of non-epithelial tissue, UBM, SIS, and UBS.

2. The method of claim 1 wherein said micronized powder is non-enzymatically treated.

3. The method of claim 1 wherein said micronized powder is stored at room temperature for at least two months.

4. The method of claim 1 wherein said micronized powder is stored at room temperature for at least six months.

5. The method of claim 1 wherein the infection is selected from the group of bacteria consisting of Staphylococcus aureus, Pseudomonas aeruginosa, and Klebsiella pneumoniae.

6. The method of claim 1 wherein said infection is localized at least to the lung.

7. The method of claim 1 wherein said airway is trachea.

8. The method of claim 1 wherein said administering route is intra-tracheal or intra-nasal.

9. The method of claim 1 wherein said administering route is via inhalation.

10. The method of claim 1 wherein said administering is via a spray.

11. The method of claim 1 wherein the extracellular matrix material comprises urinary bladder matrix (UBM).

12. The method of claim 1 wherein the extracellular matrix material comprises UBS.

13. The method of claim 1 wherein said treatment comprises lavaging the airways of the patient with the micronized particle in a buffer solution.

14. A composition, comprising:
a reconstituted material in a buffer solution comprising digested, micronized powder obtained from a devitalized extracellular matrix material including epithelial basement membrane, said reconstituted material comprising one or more native components of the extracellular matrix.

15. The composition of claim 14 wherein the micronized powder is non-cross-linked.

16. A method for reducing bacterial biofilm formation in a patient infected with the bacteria, comprising:
administering to said patient a micronized, devitalized extracellular matrix of an epithelial tissue comprising bactericidal activity against one or more bacteria selected from the group consisting of MSSA-, MSRA-Staphylococcus aureus, Klebsiella pneumoniae and Pseudomonas aeruginosa.

17. The method of claim 16 wherein the micronized powder is non-cross-linked.

18. A method for protecting a mammal from a bacterial-induced infection, comprising:
providing a reconstituted material comprising a micronized powder in a buffer solution obtained from a devitalized extracellular matrix material of an epithelial tissue, said reconstituted material comprising one or more native components of the extracellular matrix; and
administering said material in a therapeutically effective dose by a route selected from the group consisting of intra-tracheal instillation, intra-nasal instillation-inhalation, spray, topical application, and combinations thereof.

19. The method of claim 18 wherein the micronized powder is non-cross-linked.
